(19) United States
(12) Patent Application Publication
(10) Pub. No.: US 2025/0275980 A1
(43) Pub. Date: Sep. 4, 2025

(54) METHYLTHIONINIUM SALT-CONTAINING COMPOSITIONS AND METHODS

(71) Applicants: Darren LOPEZ, West Valley City, UT (US); Dan SCHMIDT, Salt Lake City, UT (US)

(72) Inventors: Darren LOPEZ, West Valley City, UT (US); Dan SCHMIDT, Salt Lake City, UT (US)

(21) Appl. No.: 18/931,277

(22) Filed: Oct. 30, 2024

Related U.S. Application Data
(60) Provisional application No. 63/560,474, filed on Mar. 1, 2024.

(51) Int. Cl. A61K 31/5415; A61K 9/00; A61K 31/00; A61K 31/198; A61K 31/355; A61K 31/375; A61K 31/4415; A61K 31/455; A61K 31/465; A61K 31/522; A61K 33/32; A61K 33/38; A61K 33/40; A61K 36/67; A61K 36/82; A61P 3/04; A61P 25/28; A61P 31/14

(52) U.S. Cl. CPC A61K 31/5415; A61K 9/0053; A61K 31/198; A61K 31/355; A61K 31/375; A61K 31/4415; A61K 31/455; A61K 31/465; A61K 31/522; A61K 31/658; A61K 33/32; A61K 33/38; A61K 33/40; A61K 36/67; A61K 36/82; A61P 3/04; A61P 25/28; A61P 31/14

(57) ABSTRACT

Methylthioninium salt-containing compositions can include methylthioninium chloride (Methylene Blue) in combination with additional compounds that work cooperatively and/or synergistically to treat various conditions. In one example, the methylthioninium salt-containing composition can include methylthioninium chloride, caffeine, and L-theanine. The methylthioninium chloride and the caffeine can be present at a weight ratio from about 1:75 to about 1:4, the methylthioninium chloride and the L-theanine can be present at a weight ratio from about 1:200 to about 1:10, and the caffeine and the L-theanine can be present at a weight ratio from about 1:5 to about 1:1.

The present application claims the benefit of U.S. Provisional Patent Application No. 63/560,474, filed on Mar. 1, 2024, the entirety of which is incorporated herein by reference.

### BACKGROUND

Methylthioninium chloride, referred to conventionally as Methylene Blue, is a synthetic compound often used for treating and/or diagnosing certain medical conditions. It functions similar to hemoglobin, which is a transporter of oxygen. Methylene blue is also a powerful redox agent, which is blue when oxidized and colorless when deoxygenated. In the therapeutic space, methylene blue has been used in the treatment of psychotic disorders, but is not as effective as it could be in treating cognitive and/or other conditions as it could be if formulated with other cooperative and/or synergistic compounds.

### DETAILED DESCRIPTION

In accordance with examples of the present disclosure, methylthioninium salt-containing compositions, methods of manufacture, and methods of treatment are provided herein. The methylthioninium salt-containing compositions can be suitable for oral administration via oral ingestion (swallowing), e.g., capsule, tablet, soft-gel, powder, or liquid, e.g., solution or suspension, and/or via transmucosal oral administration by transmucosal delivery, e.g., buccal, sublabial, sublingual, etc. In some instances, oral delivery may include delivery occurring by oral ingestion with a portion being delivered by transmucosal oral delivery prior to or after swallowing the bulk of the composition, e.g., liquid suspensions may provide some transmucosal delivery due to contact with mucosal tissues within the mouth. In accordance with this, the methylthioninium salt-containing compositions of the present disclosure can include, for example, methylthioninium chloride (also known as methylene blue) along with one or more other component(s), such as caffeine, L-theanine, green tea, guarana, magnesium, vitamin C, amino acid, e.g., glycine, N-acetylcysteine (NAC), vitamin B3, e.g., niacinamide and/or nicotinic acid, black pepper, vitamin B6, manganese, cannabinoids, nicotine, etc. With specific respect to the liquid suspensions described herein, the methylthioninium salt-containing composition may include one or more of the aforementioned other component(s) and/or hydrogen peroxide, colloidal silver (or nanosilver), mineral oxides and/or halides, e.g., magnesium chloride, etc.

In accordance with this, the present disclosure is drawn to methylthioninium salt-containing compositions, which can be suitable for treating various diseases or conditions, including many cognitive conditions and conditions that impact cognition and the central nervous system (CNS). Examples include attention disorders, e.g., ADHD, ADD, etc.; metabolism GLP-1 enhancement, e.g., learning and memory disfunction; long COVID or vaccine-induced spike protein damage, e.g., nerve damage from infection-triggered immune disfunction; brain fog; Lyme disease, e.g., verbal memory, processing speed, neurodegenerative disorders, dementia, etc.; early stage cognitive decline, e.g., dementia, Alzheimer's, etc.; seizures; epilepsy; sleep disorders; psychotic disorders; mood disorders; cognitive function, e.g., memory formation, recall, etc.; anxiety; depression; cellular and/or mitochondrial function' traumatic brain injury (TBI); energy enhancement/fatigue reduction; inflammation; low cerebral blood flow; schizophrenia; etc. Various formulations described herein can be useful for treating subsets of these and other conditions associated with cognition and/or CNS function.

In some examples, methylthioninium salt-containing compositions can include methylthioninium chloride (methylene blue), caffeine, and L-theanine. The methylthioninium chloride and the caffeine can be present at a weight ratio from about 1:75 to about 1:4, the methylthioninium chloride and the L-theanine can be present at a weight ratio from about 1:200 to about 1:10, and the caffeine and the L-theanine can be preset at a weight ratio from about 1:5 to about 1:1. This methylthioninium salt-containing composition can be used for any of the various conditions described herein, but in particular can be particularly useful when orally administered to a subject to treat attention disorders, such as attention deficit hyperactivity disorder (ADHD) or attention deficit disorder (ADD). This particular composition can also be used in supporting weight loss as well as for metabolism glucagon-like peptide-1 (GLP-1) enhancement.

In other examples methylthioninium salt-containing compositions can be prepared as a liquid suspension (for oral delivery) including water, methylthioninium chloride, mineral oxides and/or chlorides, and hydrogen peroxide. The methylthioninium chloride and the mineral oxides and/or chlorides can be present at a weight ratio of about 100:1 to about 1:4. This methylthioninium salt-containing composition can be used for any of the various conditions described herein, but in particular can be particularly useful when orally administered to a subject to treat long COVID or vaccine-induced spike protein damage, brain fog, or Lyme disease.

In other examples, methylthioninium salt-containing compositions can include methylthioninium chloride and vitamin C at a weight ratio from about 1:2.5 to about 1:7.5. This methylthioninium salt-containing composition can be used for any of the various conditions described herein, but in particular can be particularly useful when orally administered to a subject to treat early stage cognitive decline, e.g., dementia, Alzheimer's disease, etc.

In other examples, methylthioninium salt-containing compositions can include methylthioninium chloride, glycine, N-acetylcysteine, and a vitamin B3. The methylthioninium chloride and the glycine can be present at a weight ratio from about 1:3 to about 1:25, the methylthioninium chloride and the N-acetylcysteine can be present at a weight ratio from about 1:3 to about 1:25, and the methylthioninium chloride and the vitamin B3 can be present at a weight ratio from about 1:2 to about 1:12. This methylthioninium salt-containing composition can be used for any of the various conditions described herein, but in particular can be particularly useful when orally administered to a subject to ameliorate general cognitive decline and/or to treat traumatic brain injury or pre-emptively treat general cognitive decline.

In other examples, methylthioninium salt-containing compositions can include methylthioninium chloride, a cannabinoid, vitamin B6, vitamin E, and manganese. The methylthioninium chloride and the cannabinoid can be present at a weight ratio from about 1:3 to about 1:10, the methylthioninium chloride and the vitamin B6 can be present at a weight ratio from about 10:1 to about 2:1, the methylthioninium chloride and the vitamin E can be present at a weight ratio from about 1:3 to about 3:1, and the methylthioninium chloride and the manganese metal content can be present at a weight ratio from about 10:1 to about 2:1. This methylthioninium salt-containing composition can be used for any of the various conditions described herein, but in particular can be particularly useful when orally administered to a subject to treat seizures, epilepsy, and sleep disorders, e.g., for sleep support.

In another example, a combination therapy can include i) co-administering two or more methylthioninium salt-containing compositions or ii) administering a composition including all compositional components of the two or more methylthioninium salt-containing compositions to a subject. The two or more methylthioninium salt-containing compositions can be independently selected from the group consisting of: MB1 which includes methylthioninium chloride, caffeine, and L-theanine; MB2 which includes methylthioninium chloride green tea, guarana, and L-theanine; MB3 which includes methylthioninium chloride, mineral oxides/chlorides, and hydrogen peroxide; MB4 which includes methylthioninium chloride, mineral oxides/chlorides, hydrogen peroxide, and colloidal silver; MB5 which includes methylthioninium chloride and vitamin C; MB6 which includes methylthioninium chloride, glycine, N-acetylcysteine, and vitamin B3; and MB7 which includes methylthioninium chloride, CBD, vitamin B6, vitamin E, and manganese.

It is noted that when discussing the methylthioninium salt-containing compositions or any of the related methods herein, discussions of one type of example are considered applicable to other examples whether or not they are explicitly discussed in the context of that example unless expressly indicated otherwise. Thus, for example, when discussing caffeine in the context of one or more of the methylthioninium salt-containing compositions, such disclosure is also relevant to and directly supported in context of other composition examples as well as any of the methods described herein, and vice versa. Furthermore, for simplicity and illustrative purposes, numerous specific details are set forth in order to provide a thorough understanding of the present disclosure. It will be readily apparent however, that the present disclosure can be practiced without limitation to some of these specific details. In other instances, certain methods, systems, materials, and structures have not been described in detail so as not to obscure the present disclosure.

Furthermore, terms used herein will have their ordinary meaning in the relevant technical field unless specified otherwise. In some instances, there are terms defined more specifically throughout the specification, with a few more general terms included at the end of the specification. These more specifically defined terms have the meaning as described herein.

### Methylthioninium Salt-Containing Compositions

### Methylthioninium Chloride, Caffeine, and L-Theanine

As mentioned, in some examples, methylthioninium salt-containing compositions can include methylthioninium chloride (methylene blue), caffeine, and L-theanine. The methylthioninium chloride and the caffeine can be present at a weight ratio from about 1:75 to about 1:4, from about 1:50 to about 1:5, from about 1:35 to about 1:10, or from about 1:25 to about 1:15. The methylthioninium chloride and the L-theanine can be present at a weight ratio from about 1:200 to about 1:10, from about 1:100 to about 1:15, from about 1:75 to about 1:20, or from about 1:50 to about 1:25. The caffeine and the L-theanine can be preset at a weight ratio from about 1:5 to about 1:1, from about 1:4 to about 1:1, from about 1:3 to about 1:1.5, or about 2:1.

The combination of methylthioninium chloride, caffeine, and L-theanine provides some unique properties when co-formulated together, which can enhance or expand the benefits of the individual ingredients if dosed alone. For example, while caffeine is a stimulant, L-theanine promotes relaxation and reduces stress levels. In the body, with both present at appropriate concentrations, these two compounds can balance out the effects of one another, increasing the productivity and improve mental clarity of a subject taking this combination. Thus, the subject can experience the effects of the stimulant without all of the negative side effects that are sometimes associated with caffeine consumption, e.g., jitters, etc. Additionally, stacking caffeine and L-theanine can work synergistically to increase performance, reduce anxiety, etc., even when performing stressful tasks. In some examples, the caffeine to L-theanine ratio can be about 2:1 for more optimal performance, but ratios outside of this range can likewise be used, as described above. Furthermore, the presence of the methylene blue at a relatively low dose provides the additional benefit of increasing oxygen to the body, and in particular the central nervous system, e.g., brain, as it is capable of passing the blood brain barrier. Furthermore, the methylene blue can assist with preventing dopamine and/or serotonin from breaking down. Thus, increased levels of neurotransmitters can be present, enhancing cognitive performance, memory, focus, learning, mood, and overall well-being.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 1 mg to about 12 mg of methylthioninium chloride, from about 40 mg to about 225 mg of caffeine, and from about 50 mg to about 450 mg of L-theanine. In a more specific example, a typical dose of the methylthioninium salt-containing composition can include from about 1.5 mg to about 5 mg of methylthioninium chloride, from about 25 mg to about 125 mg of caffeine, and from about 50 mg to about 250 mg of L-theanine. In a more specific example, the methylthioninium salt-containing composition can include from about 2 mg to about 4 mg of methylthioninium chloride, from about 25 mg to about 75 mg of caffeine, and from about 50 mg to about 150 mg of L-theanine. Higher daily intakes of some or all of these compounds can likewise be taken in divided doses throughout the day in some examples. For example, depending on the treatment, oral delivery can be used to treat a subject via from about 1 to about 4 dosages per day.

The caffeine and/or the L-theanine can be provided as pure substances, such as extracts in order to generate more precise dosing, but in some examples, the caffeine and/or the L-theanine can be provided by certain natural components that contain caffeine and/or L-theanine (as well as other compounds that may also be present in the natural components). For example, in some examples, at least a portion of the caffeine and L-theanine can be provided by green tea and/or at least a portion of the caffeine can be provided by guarana. Example methylthioninium salt-containing compositions containing green tea and/or guarana may contain a weight ratio of methylthioninium chloride to green tea from about 1:25 to about 1:3, from about 1:20 to about 1:4, from about 1:15 to about 1:5, or about 1:10. These compositions may likewise include a methylthioninium chloride to guarana weight ratio from about 1:10 to about 1:40, from about 1:15 to about 1:35, or from about 1:20 to about 1:30, for example. The weight ratio of methylthioninium chloride to L-theanine can be from about 1:200 to about 1:10, from about 1:100 to about 1:15, from about 1:75 to about 1:20, or from about 1:50 to about 1:25. Notably, if green tea is present, at least some of the L-theanine may be provided by the presence of the green tea, for example. Furthermore, in some examples, if both green tea and guarana are present, they can be present at a weight ratio from about 1:5 to about 1:1, from about 1:4 to about 1:1.5, or from about 1:3 to about 1:2.

As an oral dosage form, this methylthioninium salt-containing composition that includes green tea and/or guarana to provide at least a portion of its caffeine component can be prepared in the form of tablets, capsules, soft-gels, powders, liquid suspensions or solutions, or transmucosal oral formulations. An example of a typical dose of the methylthioninium salt-containing composition can include from about 1 mg to about 12 mg of methylthioninium chloride, from about 10 mg to about 120 mg green tea, from about 20 mg to about 220 guarana, and from about 50 mg to about 450 mg of L-theanine. In a more specific example, a typical dose of the methylthioninium salt-containing composition can include from about 1.5 mg to about 5 mg of methylthioninium chloride, from about 15 mg to about 75 mg of green tea, from about 40 mg to about 100 mg guarana, and from about 50 mg to about 250 mg of L-theanine. In a more specific example, the methylthioninium salt-containing composition can include from about 2 mg to about 4 mg of methylthioninium chloride, from about 20 mg to about 50 mg of green tea, from about 50 mg to about 75 mg of guarana, and from about 50 mg to about 150 mg of L-theanine. Depending on the treatment, oral delivery can be used to treat a subject via from about 1 to about 4 dosages per day, for example.

These methylthioninium salt-containing compositions can be used for any of the various conditions described herein, but can be particularly useful when orally administered to a subject to treat attention disorders, such as attention deficit hyperactivity disorder (ADHD) or attention deficit disorder (ADD), for example. This particular composition can also be used in supporting weight loss as well as for metabolism glucagon-like peptide-1 (GLP-1) enhancement, and can also improve insulin sensitivity and enhance mitochondria health. In further detail, these combinations of methylthioninium salt-containing compositions can be used for metabolic ignition, promoting calorie burning and increased energy expenditure, which can be particularly the case in compositions containing the guarana. In fact, the synergy between mitochondrial support provided by methylthioninium chloride and the natural caffeine and other components of guarana can boost mitochondrial health, aid in efficient nutrient utilization, and foster overall cell vitality. On the other hand, green tea provides a good source of catechins, such as EGCG, and works synergistically with guarana to even further augment weight loss, stimulate fat oxidation, regulate appetite, improve insulin sensitivity, etc. The thermogenic properties of green tea combined with the metabolic boost provided by guarana can promote efficient fat utilization, calorie burning, and a more streamlined metabolism, e.g., thermogenesis and fat utilization. In further detail, both guarana and green tea work synergistically together to combat oxidative stress due to their individual unique antioxidant properties, providing an oxidative stress defense that is better than either compound when used alone. In other examples, pairing caffeine (such as from guarana) with L-theanine, can work synergistically together to provide an energy boost accompanied by mental clarity and focus. The L-theanine can also reduce the jittery effect of caffeine that some subjects experience.

In further detail, the methylthioninium salt-containing compositions can include additional compounds, including any of the other compounds described herein, and in some more specific examples, may further include black pepper, vitamin B6, vitamin E, manganese, a cannabinoid, nicotine, or a combination thereof.

In accordance with this, a method of treating a subject can include orally administering the methylthioninium salt-containing composition described herein which includes methylthioninium chloride, caffeine, and L-theanine. The caffeine may, in some examples, be provided in part or in whole by green tea and/or guarana. These methylthioninium salt-containing compositions can be used for any of the various conditions described herein, but can be particularly useful when orally administered to a subject to treat attention disorders, such as attention deficit hyperactivity disorder (ADHD) or attention deficit disorder (ADD), for example. They can also be used to ameliorate brain fog or dementia, and/or can be used to treat Lyme disease or chronic fatigue, for example.

With more specific respect to long COVID or vaccine-induced spike protein damage and/or dementia, the one or more of the methylthioninium salt-containing compositions described herein can be administered to reduce symptoms and/or heal the body and/or brain. As an example, a composition that includes methylthioninium chloride, caffeine, and L-theanine can be taken first thing in the morning to jump start the brain. Other methylthioninium salt-containing compositions described also herein can likewise be administered, such as the composition that includes methylthioninium chloride and vitamin C (described hereinafter), or the composition that includes methylthioninium chloride, glycine, N-acetylcysteine, and niacinamide (also described hereinafter), among others. In some examples, when treating long COVID or vaccine-induced spike protein damage in particular, the subject may benefit initially from the boost of energy in the morning from one or more of these methylthioninium salt-containing compositions in combination with a composition that includes methylthioninium chloride, mineral oxides/chlorides, and hydrogen peroxide (described hereinafter), which may further be formulated to also include colloidal silver in some examples.

### Methylthioninium Chloride, Mineral Oxides/Chlorides, and Hydrogen Peroxide

As mentioned, in some examples, methylthioninium salt-containing compositions can be in the form of a liquid suspension (for oral delivery), and can include water, methylthioninium chloride, mineral oxides and/or chlorides, and hydrogen peroxide. The methylthioninium chloride and the mineral oxides and/or chlorides can be present at a weight ratio from about 100:1 to about 1:4, from about 40:1 to about 2:1, from about 30:1 to about 3:1, or from about 25:1 to about 5:1.

As an example, a single dose of this particular methylthioninium salt-containing composition, e.g., dosed at about 0.25 mL to about 5 mL or about 0.5 mL to about 2.5 mL, can include from about 1 mg to about 20 mg of the methylthioninium chloride per mL and from about 0.2 mg to about 10 mg of the mineral oxides and/or chlorides per milliliter (mL). Higher daily intakes of some or all of these compounds can likewise be taken in divided doses throughout the day in some examples. The mineral oxides and/or chlorides in some examples may include magnesium chloride, potassium chlorate, sodium chloride, and/or magnesium oxide. In some examples, the methylthioninium chloride and the mineral oxides and/or chlorides (in combination) may be present at from about 0.01 wt % to about 0.5 wt %, from 0.025 wt % to about 0.05 wt % to about 0.3 wt %, or from about 0.1 wt % to about 0.3 wt %. The magnesium chloride and/or the sodium chloride, for example, can provide the benefits of a digestive aid, as the chloride is used in the stomach in the formation of stomach acid, e.g., HCl. The potassium chlorate can contribute as a strong oxidizer and can benefit oral health care, for example. The magnesium oxide can provide the benefits of an antacid compound.

In further detail, these methylthioninium salt-containing compositions can include additional compounds, including any of the other compounds described herein, and in some more specific examples, may further include colloidal silver, e.g., from about 5 ppm to about 50 ppm or from about 10 ppm to about 40 ppm with a particle size from about 3 nm to about 20 nm or from about 5 nm to about 15 nm. Other additional compounds that can be present include black pepper, vitamin B6, vitamin E, manganese, a cannabinoid, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

In accordance with this, a method of treating a subject can include orally administering the methylthioninium salt-containing composition described herein which includes water, methylthioninium chloride, mineral oxides and/or chlorides, and hydrogen peroxide. These methylthioninium salt-containing compositions can be used for any of the various conditions described herein, but in particular can be particularly useful when orally administered to a subject, e.g., typically daily, to treat long COVID or vaccine-induced spike protein damage, ameliorate brain fog, treat Lyme disease, and/or some of the other conditions outlined herein.

### Methylthioninium Chloride and Vitamin C

In some examples, the methylthioninium salt-containing compositions can include methylthioninium chloride and vitamin C (ascorbic acid and/or ascorbate), which can be present at a weight ratio from about 1:2.5 to about 1:7.5 or from about 1:4 to about 1:6.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 7.5 mg to about 25 mg or from about 10 mg to about 20 mg of methylthioninium chloride, and from about 50 mg to about 150 mg, from about 50 mg to about 100 mg, or from about 60 mg to about 90 mg of the vitamin C (including ascorbic acid and/or ascorbate). Higher daily intakes of some or all of these compounds can likewise be taken in divided doses throughout the day in some examples.

In further detail, these methylthioninium salt-containing compositions can include additional compounds, including any of the other compounds described herein, and in some more specific examples, may further include black pepper, vitamin B6, vitamin E, manganese, a cannabinoid, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

In accordance with this, a method of treating a subject can include orally administering the methylthioninium salt-containing composition described herein which includes methylthioninium chloride and vitamin C. These methylthioninium salt-containing compositions can be used for any of the various conditions described herein, but can be particularly useful when orally administered to a subject, e.g., typically daily, to treat early stage cognitive decline, e.g., dementia, Alzheimer's disease, etc.

In some examples, the methylthioninium salt-containing composition containing vitamin C can be used for weight loss support, and may include coadministration with a GLP-1 medication (or agonist), such as dulaglutide, exenatide, liraglutide, lixisenatide, semaglutide, tirzepatide, or the like. GLP-1 agonists can be used to treat diabetes or subjects that may be pre-diabetic and/or overweight by stimulating the release of insulin and/or lower blood sugar levels. By combining a GLP-1 agonist with this or other methylthioninium salt-containing compositions described herein, additional weight loss along with enhanced energy may be experienced, for example.

### Methylthioninium Chloride, Glycine, N-Acetylcysteine, and Vitamin B3

In some examples, the methylthioninium salt-containing compositions can include methylthioninium chloride, glycine, N-acetylcysteine, and a vitamin B3. In this example, the methylthioninium chloride and the glycine can be present at a weight ratio from about 1:3 to about 1:25 or from about 1:5 to about 1:15, the methylthioninium chloride and the N-acetylcysteine can be present at a weight ratio from about 1:3 to about 1:25 or from about 1:5 to about 1:15, and/or the methylthioninium chloride and the vitamin B3 can be present at a weight ratio from about 1:2 to about 1:12 or from about 1:2 to about 2:1. In a more detailed example, the weight ratio of glycine to N-acetylcysteine can be from about 1:3 to about 3:1, from about 1:2 to about 2:1, or about 1:1. The vitamin B3, for example, can be in the form of nicotinic acid (flushing-type niacin) and/or niacinamide. In some examples, the vitamin B3 consists or consists essentially of niacinamide.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 2 mg to about 15 mg or from about 2.5 mg to about 10 mg of methylthioninium chloride. Thus, for example, a single dose may be about 5 mg. However, multiple “single” doses can be administered throughout the day, or this formulation can be stacked with other methylthioninium salt-containing compounds described herein. In further detail, the methylthioninium salt-containing composition can include from about 20 mg to about 100 mg or from about 20 mg to about 75 mg glycine, from about 25 mg to about 100 mg or from about 25 mg to about 75 mg N-acetylcysteine, and/or from about 5 mg to about 50 mg or from about 15 mg to about 40 mg of vitamin B3, e.g., niacinamide. Higher daily intakes of some or all of these compounds can likewise be taken in divided doses throughout the day in some examples.

In further detail, these methylthioninium salt-containing compositions can include additional compounds, including any of the other compounds described herein, and in some more specific examples, may further include black pepper, vitamin B6, vitamin E, manganese, a cannabinoid, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

A method of treating a subject can include orally administering the methylthioninium salt-containing composition described herein which includes methylthioninium chloride, glycine, N-acetylcysteine, and vitamin B3. These methylthioninium salt-containing compositions can be used for any of the various conditions described herein, but can be particularly useful when orally administered to a subject, e.g., typically daily, to treat cognitive conditions, such as those related to memory, behavior, thinking, etc. Examples of conditions for which this more specific composition can be beneficial include the treatment/amelioration of general cognitive decline (pre-emptively or after onset) and/or the treatment of traumatic brain injury. In further detail, as the methylthioninium chloride is a strong antioxidant, it can be dosed to reduce oxidative stress, enhance energy levels, and improve cognition. Methylthioninium chloride can also protect the brain from damage caused by beta-amyloid, a protein that may contribute to the development of Alzheimer's disease. In further detail, glycine, which is the simplest amino acid, can also have neuroprotective properties and can assist with improving cognition, also assisting with the prevention of the formation of beta-amyloid plaques. Furthermore, vitamin B3, and particularly niacinamide in some examples, can be protective of the central nervous system, e.g., brain and nervous system, including protection against beta-amyloid accumulation and other harmful brain changes associated with dementia. Thus, methylthioninium chloride, the glycine, and the vitamin B3 can work together at least in the protection against beta-amyloid accumulation, for example. Furthermore, N-acetylcysteine is also a powerful antioxidant effective at protecting the central nervous system, with supplementation providing improved cognition, memory, etc., particularly with subjects suffering from one form or another of dementia.

### Methylthioninium Chloride, Cannabinoids, Vitamin B6, Vitamin E, and Manganese

In some examples, the methylthioninium salt-containing compositions can include methylthioninium chloride, a cannabinoid, vitamin B6, vitamin E, and manganese. In some examples, the methylthioninium chloride and the cannabinoid can be present at a weight ratio from about 1:3 to about 1:10 or from about 1:4 to about 1:7, the methylthioninium chloride and the vitamin B6 can be present at a weight ratio from about 10:1 to about 2:1 or from about 7:1 to about 3:1, the methylthioninium chloride and the vitamin E can be present at a weight ratio from about 1:3 to about 3:1 or from about 1:2 to about 1:1, and the methylthioninium chloride and the manganese metal content can be present at a weight ratio from about 10:1 to about 2:1 or from about 7:1 to about 2.5:1.

As an oral dosage form, this composition can be prepared in the form of tablets, capsules, soft-gels, powders, liquid suspensions or solutions, or transmucosal oral formulations. An example of a typical dose of the methylthioninium salt-containing composition can include from about 1 mg to about 12 mg or from about 2 mg to about 10 mg of methylthioninium chloride, from about 10 mg to about 50 mg or from about 15 mg to about 40 mg of the cannabinoid(s), from about 0.2 mg to about 3 mg or from about 0.5 mg to about 2 mg of the vitamin B6, from about 3 mg to about 15 mg or from about 5 mg to about 10 mg of the vitamin E, and/or from about 0.5 mg to about 4 mg or from about 1 mg to about 3 mg of the manganese (by metal content). Again, higher daily intakes of some or all of these compounds can likewise be taken in divided doses throughout the day in some examples.

These compositions can be effective for treating/preventing epilepsy and/or seizures, and can also be effective for promoting or treating sleep disorders, for example. The methylthioninium chloride, for example, can act as a redox agent and mitochondrial enhancer. By improving mitochondrial function and energy production in neurons, it enhances their resilience to excitotoxicity and oxidative stress, both implicated in epilepsy. Methylthioninium chloride also has ion channel-blocking properties, potentially stabilizing neuronal membranes and reducing hyperexcitability. The cannabinoid(s), e.g., CBD, can interact with the endocannabinoid system, modulating neurotransmitter release and ion channel activity, which can reduce neuronal hyperexcitability associated with seizures.

Furthermore, the cannabinoid CBD can be anti-inflammatory and neuroprotective, which can assist with mitigating seizure activity. Vitamin B6, particularly in the form of pyridoxal phosphate (PLP), is a cofactor for enzymes involved in neurotransmitter synthesis and metabolism, such as glutamate decarboxylase, which converts glutamate to GABA, an inhibitory neurotransmitter. Adequate levels of vitamin B6 can support GABAergic transmission, helping to dampen excessive neuronal firing and reduce seizure susceptibility. Furthermore, vitamin E is a potent antioxidant that scavenges free radicals and protects cell membranes from oxidative damage. Oxidative stress is implicated in epileptogenesis, and the presence of vitamin E in the compositions described herein can assist with mitigating the effects and reducing the frequency of seizures. Furthermore, manganese is a cofactor for various enzymes, including glutamine synthetase, which converts glutamate to glutamine. Glutamine assists with maintaining the glutamate-glutamine cycle and regulating glutamate levels in the brain. Dysregulation of glutamate homeostasis is associated with neuronal hyperexcitability and seizures. Adequate manganese levels support this cycle, potentially reducing glutamate-mediated excitotoxicity and seizure occurrence. By combining these components in a single formulation or composition for orally dosing subjects can promote a synergistic effect in epilepsy management, with the cannabinoid CBD and methylthioninium chloride targeting hyperexcitability through alternative pathways (both compounds having a different mechanism of action and thus a stronger effect). Furthermore, the presence of vitamins B6 and E, along with manganese, can support neurotransmitter balance (vitamin B6), and can mitigate oxidative stress (vitamin E, and regulate glutamate levels (manganese), thus providing a more effective approach for managing epilepsy and seizures than any of these compounds alone in most instances.

In further detail, these methylthioninium salt-containing compositions can include additional compounds, including any of the other compounds described herein, and in some more specific examples, may further include black pepper, nicotine, or a combination thereof. Other ingredients that may be present include caffeine, L-theanine, or a combination thereof. If caffeine is included, it can be provided by the purified compound, green tea, guarana, and/or other compositions rich in caffeine. Additionally, if L-theanine is included in this composition, it can be provided by the purified compound, guarana, and/or other compositions rich in L-theanine.

In accordance with this, a method of treating a subject can include orally administering the methylthioninium salt-containing composition described herein which includes methylthioninium chloride, a cannabinoid, vitamin B6, vitamin E, and manganese. These methylthioninium salt-containing compositions can be used for any of the various conditions described herein, but can be particularly useful when orally administered to a subject, e.g., typically daily, to treat/prevent seizures, epilepsy, and sleep disorders, e.g., provide sleep support, as described above.

### Combination Therapies

In accordance with examples of the present disclosure, multiple methylthioninium salt-containing compositions can be combined to achieve specific enhanced effects. For example, MB1-MB7 are presented generally below in Table 1, which illustrates the core active compounds that can be present in these compositions, as follows:

TABLE 1

| Composition ID | Ingredients |
|---|---|
| MB1 | MB^{†}, caffeine, L-theanine |
| MB2 | MB^{†}, green tea, guarana, L-theanine |
| MB3 | MB^{†}, mineral oxides/chlorides, hydrogen peroxide |
| | (formed in situ) |
| MB4 | MB^{†}, mineral oxides/chlorides, hydrogen peroxide, |
| | colloidal silver |
| MB5 | MB^{†}, vitamin C |
| MB6 | MB^{†}, glycine, N-acetylcysteine, vitamin B3 |
| | (niacinamide) |
| MB7 | MB^{†}, cannabidiol (CBD), vitamin B6, vitamin E, |
| | manganese |

MB^{†} refers to methylthioninium chloride in these examples.

In accordance with some examples, any two (or more) compositions presented in Table 1 may be combined for combination therapy, or in some examples, may be combined into a single dosage form. In some examples, when doing combination therapy, the daily dose of the methylthioninium salt (from the multiple compositions or combined dosage form) can be up to about 50 mg, up to about 40 mg, up to about 30 mg, up to 20 mg, or up to 10 mg per day. Thus, the total amount of methylthioninium chloride per day may be from about 2 mg to about 50 mg, from about 4 mg to about 50 mg, from about 5 mg to about 50 mg, from about 2 mg to about 40 mg, from about 4 mg to about 40 mg, from about 5 mg to about 40 mg, or from about 4 mg to about 25 mg, for example. Combination therapies (or single formulations including compounds from multiple compositions) can include combining MB1 or MB2 with any one (or two) of MB3-MB7, e.g., combining MB1 or MB2 with MB3 or MB4, combining MB1 or MB2 with MB5, combining MB1 or MB2 with MB6, or combining MB1 or MB2 with MB7. In other examples, combinations can include combining MB3 or MB4 with one or more of MB5-MB7, e.g., combining MB3 or MB4 with MB5, combining MB3 or MB4 with MB6, or combining MB3 or MB4 with MB7. In still other examples, combinations can include combing MB5 with MB6 or combing MB5 with MB7. In further detail, combinations can include combining MB6 with MB7. There are a variety of reasons why two different methylthioninium salt-containing compositions can be combined (either as combination therapy or as a formulation containing ingredients from multiple compositions). For example, MB3 or MB4 could be combined with MB5 for treating SARS-CoV-2 (COVID-19) or other viruses, e.g., influenza, etc., treating long COVID, treating subjects for vaccine injury, e.g., vaccine-induced spike protein damage, etc. The presence of the vitamin C may assist with treating these or other conditions. Likewise, combing MB7 with MB1-MB6 could be beneficial for treating seizure and/or epilepsy. These and other combinations can likewise be beneficial for treating any of a number of conditions, including the attention deficit conditions, brain fog, Lyme disease, chronic fatigue, obesity (weight loss support), cognitive decline, e.g., dementia, Alzheimer's disease, traumatic brain injury, seizures, epilepsy, sleep disorders (sleep support), etc., as well as any of the other conditions described herein.

### Active Ingredients or Compounds

### Methylthioninium Salts (e.g., Methylthioninium Chloride or Methylene Blue)

Methylthioninium salts, such as methylthioninium chloride or methylene blue, are synthetic chemical compounds with a specific crystalline structure appearing dark green as a powder at room temperature and blue when mixed in water. The blue color can be lost with time when at rest, e.g., in its reduced state due to lack of interaction with oxygen), but re-emerges as blue when agitated, e.g., in its oxidized state. Because of these unique properties, it has been used diagnostically to distinguish tissues and fluids in a lab setting. Methylthioninium salts can also function similarly to hemoglobin, the substance responsible for transporting oxygen to the body's tissues and organs, making it a good candidate for various therapies, though its use orally over the years has led to different levels of success. In further detail, methylthioninium salt is a formal derivative of phenothiazine, with the hydrated form having three (3) molecules of water per single molecule of the methylthioninium salt compound. The structure of methylthioninium chloride is shown by way of example in Formula I, as follows:

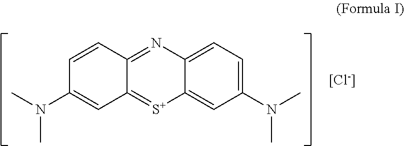

In accordance with the present disclosure, when the methylthioninium salt is admixed with various other compounds, such as caffeine, L-theanine, green tea, guarana, magnesium, vitamin C, amino acid, e.g., glycine, N-acetylcysteine (NAC), vitamin B3, e.g., niacinamide and/or nicotinic acid, black pepper, vitamin B6, manganese, cannabinoids, nicotine, and/or others, the efficacy of the methylthioninium salt can be enhanced, particularly when the methylthioninium salt is given orally at a relatively low dose, and furthermore, is combined with the other compound(s) at appropriate weight ratios. In some instances, the multiple compounds can exhibit synergy in treating various conditions, including any of a number of cognitive and/or neurological conditions.

In relation to this, it is understood that mitochondria are the energy producers of the cells, and often times, neurodegenerative and psychiatric disorders can lead to mitochondrial damage. The neuroprotective properties of methylthioninium salts can, for example, improve mitochondrial function such that a subject may see improvements in memory retention, cytochrome oxidase activity, ATP generation, etc., thus assisting with the maintenance of neural health and cognitive function. Furthermore, mitochondrial dysfunctions can decrease energy transfer to the cells resulting in cellular damage and/or death, e.g., cellular damage can increase neuroinflammation. Furthermore, when mitochondria dysfunctions, energy transfers may cease to occur, leading to cellular death. Additionally, toxic build up can occur, leading to oxidative stress and the release of free radicals (and ultimately, loss of neurons). Methylthioninium salt, such as methylene blue, can provide some alternative energy transfer that is effective for protecting neurons by increasing ATP production (which provides energy for the brain via mitochondria respiration). More specifically, as there is a link between methylthioninium salt and neurodegenerative disorders and/or energy metabolism, oral delivery of methylthioninium salt can result in electrons being received from nicotinamide adenine dinucleotide (NADH and/or NAD+, but referred to generally as “NAD”) in the presence of Complex 1 and donate them to Cytochrome C, thus providing an alternative electron transfer pathway. This can result in an increase in oxygen consumption, a decrease in glycolysis, and an increase in glucose uptake enhancing cerebral blood flow. This can be particularly useful for therapeutic uses that increase ATP production after passing through the blood-brain barrier.

In further detail, methylthioninium chloride, particularly when dosed with some of the other secondary compounds described herein, can act to enhance the depletion of dopamine common in many central nervous system conditions, such as Parkinson's disease. Furthermore, methylthioninium chloride can provide the benefits of enhancing neuroprotection within the central nervous system acting as a superoxide (antioxidant) to reduce or eliminate free radicals, reducing cerebral ischemia reperfusion damage in a transient focal cerebral ischemia model, reducing formation of amyloid plaques and neurofibrillary tangles, and repairing impairments in mitochondrial function and cellular metabolism, e.g., reducing plaque formation, etc. In short, as the brain uses a disproportionate amount of oxygen and sugar (ATP) compared to its size, orally treating a subject with a condition associated with the central nervous system can provide some of the raw materials used for proper brain function.

In accordance with examples of the present disclosure, the methylthioninium salt-containing compositions of the present disclosure all include methylthioninium chloride, which is a hormetic substance, meaning that it is most effective at lower dosages, and in fact, may be counterproductive if dosed at too high of a level. In accordance with this, the methylthioninium chloride can be present in a single dose (or total daily dose) of one or more of the methylthioninium salt-containing compositions at from about 1 mg to about 50 mg, from about 1 mg to about 25 mg, from about 1 mg to about 20 mg, from about 1 mg to about 15 mg, from about 1 mg to about 12 mg, from about 2 mg to about 20 mg, from about 2 mg to about 15 mg, from about 2 mg to about 12 mg, or from about 2 mg to about 10 mg. In some examples, a single dosage form, e.g., a single tablet, of the methylthioninium salt-containing composition can include about 1 mg to about 25 mg, from about 1 mg to about 20 mg, from about 1 mg to about 12 mg, from about 1 mg to about 6 mg, from about 6 mg to about 12 mg, from about 1 mg to about 3 mg, from about 3 mg to about 6 mg, from about 6 mg to about 9 mg, from about 9 mg to about 12 mg, or from about 12 mg to about 25 mg.

These daily dosage (taken at one time or as divided daily dosages) or single dosage form, e.g., single table, capsule, etc., ranges recited above can be used in any of the combination therapies or treatments described by way of example herein, irrespective of the narrower ranges provided for the methylthioninium salt that may be recited hereinafter in the context of more specific examples. Regarding the ratio of the methylthioninium salt to the other compounds for coadministration or in forming the methylthioninium salt-containing compositions may depend on the other compound(s) present, as illustrated by example in greater detail hereinafter.

### Caffeine

Caffeine is a compound that can be used to stimulate the central nervous system (CNS), and can also increase the production of dopamine, which is a neurochemical heavily involved in controlling concentration and focus. In additional detail, caffeine is chemically similar to nucleoside adenosine, which is why it can interact with the brain. Thus, caffeine can increase dopamine production and can block adenosine receptor sites. Furthermore, caffeine can constrict blood vessels and reduce blood flow. The chemical structure of caffeine is shown by way of example in Formula II, as follows:

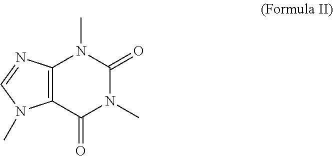

In further detail, caffeine can provide the following benefits, if dosed correctly, namely provide energy as a stimulant, boost brain health and mood due to the presence of antioxidants, improve focus and concentration, etc. Furthermore, coadministration of caffeine with L-theanine (described in greater detail below) provides a psychoactive synergistic effect promoting improved attention and alertness. Regarding dosing, the caffeine can be included in any of the methylthioninium salt-containing compositions described herein in a single dose at from about 40 mg to about 225 mg, from about 50 mg to about 200 mg, or from about 75 mg to about 150 mg, for example.

The caffeine can be added in a purified or substantially purified form, e.g., as an extract, and/or can be added via introduction with other compounds that naturally include some caffeine. Examples include green tea or guarana, both of which include a considerable concentration of caffeine along with other compounds. For example, green tea extract may contain more caffeine than green tea catechins in some instances. Green tea extract or green tea powder may include from about 15 wt % to about 20 wt % caffeine, though that depends on the extract, powder, dilution, etc. However, the benefit of using green tea as a caffeine source can be the presence of the green tea catechins, e.g., epigallocatechin (EGCG), epicatechin-3-gallate (ECG), epigallocatechin (EGC), epicatechin (EC), etc. Green tea can be included in the methylthioninium salt-containing compositions at from about 20 mg to about 300 mg, from about 20 mg to about 200 mg, or from about 20 mg to about 120 mg. The green tea catechins can be present in a single dose of the methylthioninium salt-containing compositions at from about 1 mg to about 100 mg, from about 2 mg to about 75 mg, or from about 3 mg to about 50 mg, for example.

The caffeine content provided by the green tea can be similar to that described previously unless there is a second caffeine source, e.g., guarana, in which case the total content of all caffeine sources would be calculated to range from about 40 mg to about 225 mg of caffeine content.

Guarana may be included as the sole caffeine source, or can be combined with other caffeine sources, such as green tea, purified or substantially purified caffeine, e.g. an extract, etc. Guarana is a dietary supplement or herb that is also a stimulant, and includes a considerable concentration of caffeine, e.g., about twice the content of caffeine as found in a typical coffee bean, e.g., about 2 wt % to about 8 wt % in the guarana seed. If guarana is included, it can be present at from about 50 mg to about 400 mg, from about 50 mg to about 220 mg, or from about 75 mg to about 200 mg, for example.

### L-Theanine

L-theanine is an amino acid found in nature that is not naturally produced by the body, but found in herbs and vegetables, specifically, green tea leaves, black tea leaves, and some mushrooms. As mentioned, L-theanine can be included in the methylthioninium salt-containing compositions of the present disclosure. Its presence can be even more effective when included in combination with caffeine (or a caffeine source), though formulations without both being present can also be effective for treating various conditions. L-theanine is shown by way of example in Formula III, as follows:

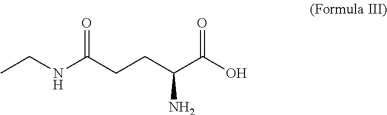

Supplementing with L-theanine can provide various benefits, including improved cognitive function, alleviation of psychological stress, maintenance of normal sleep/restful sleep, reduction of menstrual discomfort, enhanced mood, brain boosting effect, calming effect, etc. Similar to methylthioninium chloride, L-theanine can also cross the blood brain barrier, which can provide cognitive benefits and treat various disorders associated with the brain. In some examples, L-theanine can be present in a single dose of the methylthioninium salt-containing composition at from about 50 mg to about 450 mg, from about 75 mg to about 300 mg, or from about 75 mg to about 200 mg.

### Mineral Oxides/Chlorides (and Hydrogen Peroxide)

The mineral oxides and/or chlorides added to some of the methylthioninium salt-containing compositions of the present disclosure can include a variety of minerals in oxide and/or chloride form. One example is magnesium chloride, which has good bioavailability in the body. Other examples include potassium chlorate, sodium chloride, and magnesium oxide. The mineral oxides/chlorides (including chlorates) can be added, for example at from about 0.5 vol % to about 5 vol %, from about 0.5 vol % to about 3.5 vol %, from about 1 vol % to about 2.5 vol %, or at about 1.5 vol %. This liquid suspension can be orally administered as a liquid, or can be placed in one or more soft gel capsules.

When formulated in an aqueous suspension, for example, the presence of the mineral oxides and/or chlorides in combination with water and the other components can result in liberating or forming hydrogen peroxide, which can also promote several health benefits. Example health benefits include bringing additional oxygen into the body. Most diseases proliferate when there is inadequate oxygen in the body, so the presence of hydrogen peroxide being formed in an aqueous liquid suspension can likewise have healing properties related to reduction of pain, detoxification of the body, and eliminating infection, for example.

### Colloidal Silver

Colloidal silver in the form of small elemental particles or “colloids” at relatively low doses can be an effective antimicrobial and can also promote healing properties, e.g., wound healing, gut health, etc., and can support the immune system. In some examples, colloidal silver used herein can be sized in the nano-scale, e.g., up to about 50 nm or so, at a relatively low concentration, up to about 100 ppm in a liquid suspension. Sizes and concentrations above these levels can be used as well in some instances, provided issues related to bioaccumulation can be addressed. In some examples, Ag404 colloidal silver from American Biotech Labs has been shown effective for oral delivery, with an average particle size from about 5 nm to about 15 nm in size. The bioactivity of Ag404 in particular may be related at least in part to its surface area to volume ratio due to its considerably small size, without substantial bioaccumulation. Whether this colloidal silver is used or another, a benefit to using colloidal silver can relate to its ability to steal electrons from pathogens and reduce the bio load of various pathogens in the body.

### Vitamin C

Vitamin C, which can be in the form of ascorbic acid and/or an ascorbate) is a water-soluble vitamin that is found in many fruits, and is considered an essential nutrient involved in tissue repair, formation of collagen, and the enzymatic production of some neurotransmitters as part of the central nervous systems. Vitamin C is also used for the functioning of several enzymes associated with the immune system. The structure of vitamin C (as ascorbic acid) is shown in Formula IV, as follows:

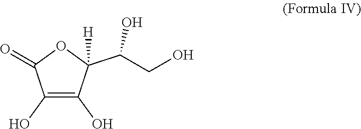 [SMILES: O=C1O[C@@H]([C@H](O)CO)C(O)=C1O]

In accordance with examples herein, vitamin C can be particularly useful when combined with methylthioninium chloride in treating disorders and diseases associated with dementia due to its enzymatic activity related to neurotransmitters. For example, vitamin C is a potent antioxidant that can help protect mitochondria from oxidative stress. Mitochondria are particularly vulnerable to oxidative damage due to the high levels of reactive oxygen species (ROS) they generate during energy production. By scavenging ROS, vitamin C helps prevent damage to mitochondrial DNA, proteins, and lipids, thus supporting their function and overall health. As methylthioninium chloride also interacts with mitochondria, e.g., it can act as an electron carrier to help with ATP production, it can work well when combined with vitamin C. When used together, vitamin C and methylthioninium chloride can provide complimentary or even synergistic effects on mitochondria health. For example, both compounds have antioxidant properties, so when used together, they may provide enhanced protection against oxidative damage to mitochondria.

### Glycine

Glycine is the simplest of all of the stable amino acids, having a single hydrogen as its “R” group (present at the carboxyl group) rather than having a longer side chain. Glycine can act as an inhibitory neurotransmitter in the right circumstances, such as in the central nervous system, e.g., spinal cord, brainstem, and retina. When glycine receptors are activated, chloride can enter the neuro via ionotropic receptors, causing inhibitory postsynaptic potential. Furthermore, glycine is a co-agonist along with glutamate for NMDA receptors. Glycine plays multiple roles in the central nervous system, but notably, when dosed properly with other compounds of the methylthioninium salt-containing compositions of the present disclosure, it can also enhance the effectiveness of certain treatments. For example, glycine can also have neuroprotective properties and can assist with improving cognition, and can also assist with the prevention of the formation of beta-amyloid plaques. The structure of glycine is shown in Formula V, as follows:

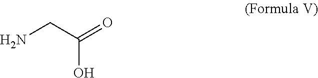

### N-Acetylcysteine (NAC)

N-acetylcysteine has many health benefits, including replenishing antioxidants, e.g., glutathione, brain health, ameliorating chronic respiratory conditions, enhancing fertility, etc.

As glutathione is one of the more bioactive antioxidants in the body, sufficient serum levels provide a good basis for neutralizing free radicals that can damage cells and tissue, With respect to central nervous system support, N-acetylcysteine can regulate glutamate levels, which is a valued neurotransmitter in the body which should be regulated within a safe range, e.g., not too much and not too little. Mental disorders, such as bipolar disorder, schizophrenia, obsessive-compulsive disorder (OCD), and substance use disorder are all conditions that can be tied to improper regulation of glutamate and/or glutathione. By replenishing glutathione and regulating glutamate in the central nervous system, brain health can be boosted. As a broad range of cognitive and behavioral systems rely on the neurotransmitter glutamate, proper serum levels of N-acetylcysteine in the body can promote cognition and behavior. In short, N-acetylcysteine is an antioxidant (and promotes the production of glutathione, which is also a powerful antioxidant) effective at protecting the central nervous system, and promoting improved cognition, memory, etc., particularly with subjects suffering from one form or another of dementia. The structure of N-acetylcysteine is shown in Formula VI, as follows:

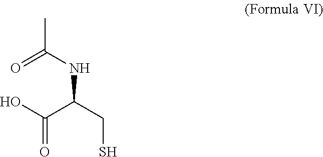

### Vitamin B

Regarding the B-complex vitamin content, any of a number of B-complex vitamins can be included in the various methylthioninium salt-containing compositions described herein. For example, the B-complex vitamins can be sourced from any of a number of liquid solutions or suspensions and/or dry B-complex vitamin sources. Example B-complex vitamins that can be included are thiamin (B1), riboflavin (B2), niacin or niacinamide (B3), pyridoxine (B6), folic acid or folate (B9), and/or cobalamin (B12). In some examples, the B-complex vitamins can be selected from vitamin B3 and/or vitamin B6. The vitamin B3, in some examples, can be in the form of niacinamide. In other examples, the vitamin B6 can be in the form of pyridoxal 5′ phosphate (PLP). In still other examples, the B-complex vitamin(s), where applicable, can be in a methylated form, which can assist with various metabolic processes. For example, methylated vitamin B9 and methylated vitamin B12 can be more bioavailable and useful when dosed appropriately, particularly with some subjects with gene mutations rendering their body inefficient for in situ methylation.

Regarding B-complex vitamins and their interaction with methylthioninium chloride, it is noted that administration of methylthioninium chloride can have a positive impact on neurodegenerative disorders and energy metabolism. With more specific reference to nicotinamide (or vitamin B3), it is noted that niacinamide can be converted to nicotinamide adenine dinucleotide (NAD, NADH), which is a versatile compound in the body. The coadministration of methylthioninium chloride with B-complex vitamins provides raw materials for establishing a pathway for the methylthioninium chloride to receive electrons from nicotinamide adenine dinucleotide (NADH) in the presence of Complex 1 and donating them to Cytochrome C. This provides an alternative electron transfer pathway causing an increase in oxygen consumption, a decrease in glycolysis and an increase in glucose uptake enhancing cerebral blood flow.

This mechanism can provide protection to neurons associated with neurodegenerative diseases by activating AMP-activated protein kinase inhibiting harmful enzymes downstream. Additionally, this can also enhance mitochondrial oxidative phosphorylation via alternative mitochondrial electron transfer, offering protection against neurodegenerative diseases. In further detail, it is understood that methylthioninium chloride crosses the brain-blood barrier easily to start ATP production. ATP provides energy for the brain through mitochondria respiration as an alternative electron carrier that transfers electrons without affecting mitochondrial activities. This increases cellular consumption rates and reduces anaerobic glycolysis, breaking down sugar (ATP) to produce energy. Rerouting mitochondrial electron transfer provides neuroprotection against chronic and acute neurological disease. Simplified, this means that methylthioninium chloride increases oxygen to the brain for the cell to breath (mitochondrial respiration) and transfers electrons to the cell to produce energy for function.

Another B-complex vitamin that can be selected for use is vitamin B6 (pyridoxine), which in some examples, can be in the form of pyridoxal 5′ phosphate (PLP). PLP is the active form of vitamin B6 and is a coenzyme for a variety of enzymatic activities, including as a significant coenzyme in transamination reactions, as well as a coenzyme in some decarboxylation, deamination, and racemization reactions of amino acids. Vitamin B6 is used by the body for normal brain development as well as keeping the nervous system and the immune system healthy. Low vitamin B6 levels can also be related to epilepsy. Additionally, deficiencies of vitamin B6 can lead to malabsorption of nutrients in the small intestine, and in some instances, can lead to conditions where healthy red blood cells cannot carry an adequate amount of oxygen to the body (including the brain and nervous system), e.g., sideroblastic anemia. This mechanism and others can cause a subject to experience confusion, depression, brain fog, and a weakened immune system. In some examples, the vitamin B6 can be coupled with one or both of vitamin B9 and/or vitamin B12, which may be in the methylated form.

### Cannabinoids

Cannabinoids are compounds derived from the cannabis plant, with the cannabinoids commonly referred to as CBD (cannabidiol) and/or THC (tetrahydrocannbidiol) receiving much of the attention. However, there are many other cannabinoids with a biogenetic origin that are also present in cannabis plants. One other notable cannabinoid that can provide health benefits is commonly referred to as CBG (cannabigerol), to name one example. It has been found that in some examples of the present disclosure, the inclusion of CBD, and in some cases CBG, can provide benefits to the methylthioninium salt-containing compositions described herein, as neither CBD nor CBG are psychoactive and both have anti-inflammatory properties and are beneficial for central nervous system health, e.g., brain health.

CBD has been shown to be effective for treating depression, panic disorders, mood disorders, psychosis, PTSD-related disorders, etc. CBD in particular produces neurotransmitters known as endocannabinoids that bind to cannabinoid receptors, and can also be neuroprotective against a variety of neurological disorders, with some data indicating efficacy against Parkinson's disease, Huntington's disease, motor disorders, e.g., cerebral palsy, etc. Regarding CBG, this compound can bind to both CB1 and CB2 receptors within the endocannabinoid system (ECS) of the body, which is responsible for maintaining homeostasis of various systems within the body via many receptor sites of the organs, central nervous system, digestive system, immune system, reproductive system, etc. With some similarities and some differences with respect to treatment efficacy of disorders related to the central nervous system, the presence of CBD and/or CBG in some instances can uniquely enhance the therapeutic properties of the methylthioninium salt-containing compositions of the present disclosure. Example central nervous system disorders where a subject may benefit from the presence of one or both of these cannabinoids include autism spectrum disorders (ASD), such as Asperger's syndrome, pervasive developmental disorder (PDD-NOS), autistic disorder, childhood disintegrative disorder, etc., as well as the various attention disorders, such as attention deficit hyperactivity disorder (ADHD), attention deficit disorder (ADD), etc. In accordance with examples herein, if both CBD and CBG are used, then they may be included in the methylthioninium salt-containing compositions at from about 1:1 to about 10:1, from about 1.5:1 to about 8:1, from about 2:1 to about 6:1, from about 2:1 to about 4:1, or from about 6.5:3 to about 7.5:3.

### Vitamin E

Vitamin E is an antioxidant that helps protect cells from damage caused by free radicals. Notably, vitamin E may have neuroprotective effects. Vitamin E is one of the fat-soluble vitamins, and as there are only a few fat-soluble vitamins, vitamin E is uniquely positioned to scavenge free radicals that can cause cellular damage, particular at locations such as the cell membrane and other locations where fats may be concentrated. Vitamin E can also enhance immune function and prevent clots from forming in the arteries of the heart. As vitamin E has neuroprotective effects and is a good lipophilic free radical scavenger, it can complement the actions of methylene blue in the context of treating neurodegenerative diseases or conditions involving oxidative stress.

### Manganese

Manganese is an essential mineral that plays a role in various physiological processes, including metabolism, bone formation, and antioxidant defense. Manganese antioxidant properties can be used, for example, to enhance the overall antioxidant capacity when used alongside an appropriate dose of methylthioninium chloride and other compounds that may be present. As the human body does not make manganese, it is often provided by food and/or supplementation, as it is responsible for breaking down carbohydrates, protein, cholesterol, etc., as well as assisting enzymes in building bones. In certain examples, the manganese can be provided by a mineral source such as manganese citrate, manganese gluconate, manganese ascorbate, or manganese amino acid chelate. Typically, when calculating the concentration of manganese in a composition, it is the metal content that is calculated for purposes of weight percentages and/or weight ratios with other components that may be present.

### Example Embodiments

In accordance with the disclosure herein, the following examples are illustrative of several embodiments of the present technology.

1. A methylthioninium salt-containing composition, comprising methylthioninium chloride, caffeine, and L-theanine, wherein the methylthioninium chloride and the caffeine are present at a weight ratio from about 1:75 to about 1:4 and the methylthioninium chloride and the L-theanine are present at a weight ratio from about 1:200 to about 1:10, and the caffeine and the L-theanine are present at a weight ratio from about 1:5 to about 1:1.

2. The methylthioninium salt-containing composition of example 1, wherein the methylthioninium chloride and the caffeine are present at a weight ratio from about 1:25 to 1:15.

3. The methylthioninium salt-containing composition of one of examples 1-2, wherein the methylthioninium chloride and the L-theanine are present at a weight ratio from about 1:50 to 1:25.

4. The methylthioninium salt-containing composition of one of examples 1-3, wherein the caffeine and the L-theanine are present at a weight ratio from about 1:3 to about 1:1.5.

5. The methylthioninium salt-containing composition of one of examples 1-4, wherein the composition is in an oral dosage form selected from tablet, capsule, soft-gel, powder, liquid suspension or solution, or transmucosal oral formulation.

6. The methylthioninium salt-containing composition of one of examples 1-5, comprising green tea or guarana providing at least a portion of the caffeine.

7. The methylthioninium salt-containing composition of one of examples 1-6, comprising green tea and guarana providing at least a portion of the caffeine.

8. The methylthioninium salt-containing composition of example 7, wherein the green tea and the guarana are present at a weight ratio of about 1:1.1 to about 1:5, the methylthioninium chloride and the green tea are present at a weight ratio from about 1:5 to about 1:25, and the methylthioninium chloride and the guarana are present at a weight ratio of about 1:10 to about 1:40.

9. The methylthioninium salt-containing composition of one of examples 1-8, wherein a single dose of the methylthioninium salt-containing composition includes about 1 mg to about 12 mg of methylthioninium chloride, about 40 mg to about 225 mg of caffeine, and about 50 mg to about 450 mg of L-theanine.

10. The methylthioninium salt-containing composition of one of examples 1-9, wherein a single dose of the methylthioninium salt-containing composition includes about 1 mg to about 12 mg of methylthioninium chloride, about 10 mg to about 120 mg of green tea, about 20 mg to about 220 mg of guarana, and about 50 mg to about 450 mg of L-theanine.

11. The methylthioninium salt-containing composition of one of examples 1-10, further comprising black pepper, vitamin B6, vitamin E, manganese, a cannabinoid, nicotine, or a combination thereof.

12. A method of treating a subject, comprising orally administering the methylthioninium salt-containing composition of one of examples 1-11 to the subject.

13. The method of example 12, wherein the methylthioninium salt-containing composition is dosed on a daily basis to treat attention disorders selected from attention deficit hyperactivity disorder (ADHD) or attention deficit disorder (ADD).

14. The method of example 12, wherein the methylthioninium salt-containing composition is dosed on a daily basis for supporting weight loss.

15. The method of example 12, wherein the methylthioninium salt-containing composition is dosed on a daily basis for metabolism glucagon-like peptide-1 enhancement.

16. A methylthioninium salt-containing composition, comprising a liquid suspension for oral delivery including water, methylthioninium chloride, mineral oxides and/or chlorides, and hydrogen peroxide, wherein the methylthioninium chloride and the mineral oxides and/or chlorides are present at a weight ratio of about 100:1 to about 1:4.

17. The methylthioninium salt-containing composition of example 16, further comprising colloidal silver.

18. The methylthioninium salt-containing composition of example 17, wherein the colloidal silver is present at from about 5 ppm to about 50 ppm by weight.

19. The methylthioninium salt-containing composition of example 17, wherein the colloidal silver has an average particle size from about 3 nm to about 20 nm.

20. The methylthioninium salt-containing composition of one of examples 16-19, wherein a single dose of the methylthioninium salt-containing composition is from about 0.5 mL to about 2.5 mL and includes from about 1 mg to about 20 mg of the methylthioninium chloride per mL, and from about 0.2 mg to about 10 mg of the mineral oxides and/or chlorides per mL.

21. The methylthioninium salt-containing composition of one of examples 16-20, wherein the mineral oxides and/or chlorides include magnesium chloride.

22. The methylthioninium salt-containing composition of one of examples 16-21, wherein the weight ratio of the methylthioninium chloride to the mineral oxides and/or chlorides is from about 40:1 to about 2:1.

23. The methylthioninium salt-containing composition of one of examples 16-22, further comprising black pepper, vitamin B6, vitamin E, manganese, a cannabinoid, nicotine, or a combination thereof.

24. The methylthioninium salt-containing composition of one of examples 16-23, further comprising caffeine, L-theanine, or a combination thereof.

25. A method of treating a subject, comprising orally administering the methylthioninium salt-containing composition of one of examples 16-24 to the subject.

26. The method of example 25, wherein the methylthioninium salt-containing composition is dosed on a daily basis to treat long COVID or vaccine-induced spike protein damage.

27. The method of example 25, wherein the methylthioninium salt-containing composition is dosed on a daily basis to ameliorate brain fog or dementia.

28. The method of example 25, wherein the methylthioninium salt-containing composition is dosed on a daily basis for treating Lyme disease or chronic fatigue.

29. A methylthioninium salt-containing composition, comprising methylthioninium chloride and vitamin C at a weight ratio from about 1:2.5 to about 1:7.5.

30. The methylthioninium salt-containing composition of example 29, wherein the weight ratio is from about 4:1 to about 6:1.

31. The methylthioninium salt-containing composition of one of examples 29-30, wherein a single dose of the methylthioninium salt-containing composition includes from about 7.5 mg to about 25 mg of the methylthioninium chloride and from about 50 mg to about 150 mg of the vitamin C.

32. The methylthioninium salt-containing composition of one of examples 29-31, wherein the composition is in an oral dosage form selected from tablet, capsule, soft-gel, powder, liquid suspension or solution, or transmucosal oral formulation.

33. The methylthioninium salt-containing composition of one of examples 29-32, further comprising caffeine, L-theanine, or a combination thereof.

34. The methylthioninium salt-containing composition of one of examples 29-33, further comprising black pepper, vitamin B6, vitamin E, manganese, a cannabinoid, nicotine, or a combination thereof.

35. A method of treating a subject, comprising orally administering the methylthioninium salt-containing composition of one of examples 29-34 to the subject.

36. The method of example 35, wherein the methylthioninium salt-containing composition is dosed on a daily basis to treat early stage cognitive decline.

37. The method of example 36, wherein the early stage cognitive decline is associated with dementia.

38. The method of example 36, wherein the early stage cognitive decline is associated with Alzheimer's disease.

39. The method of example 35, wherein the methylthioninium salt-containing composition is dosed on a daily basis for weight loss support.

40. The method of example 35, wherein the methylthioninium salt-containing composition is coadministered at the same time or at different times during the day with a GLP-1 enhancement medication.

41. A methylthioninium salt-containing composition, comprising methylthioninium chloride, glycine, N-acetylcysteine, and a vitamin B3, wherein the methylthioninium chloride and the glycine are present at a weight ratio from about 1:3 to about 1:25, the methylthioninium chloride and the N-acetylcysteine are present at a weight ratio from about 1:3 to about 1:25, and the methylthioninium chloride and the vitamin B3 are present at a weight ratio from about 1:2 to about 1:12.

42. The methylthioninium salt-containing composition of example 41, wherein the methylthioninium chloride and the glycine are present at a weight ratio from about 1:5 to about 1:15, and wherein the methylthioninium chloride and the N-acetylcysteine are present at a weight ratio from about 1:5 to about 1:15.

43. The methylthioninium salt-containing composition of one of examples 41-42, wherein the glycine and the N-acetylcysteine are present at a weight ratio from about 1:2 to about 2:1.

44. The methylthioninium salt-containing composition of one of examples 41-43, wherein the composition is in an oral dosage form selected from tablet, capsule, soft-gel, powder, liquid suspension or solution, or transmucosal oral formulation.

45. The methylthioninium salt-containing composition of one of examples 41-44, wherein the vitamin B3 is in the form of niacinamide, nicotinic acid, or a combination thereof.

46. The methylthioninium salt-containing composition of one of examples 41-45, further comprising caffeine, L-theanine, or a combination thereof.

47. The methylthioninium salt-containing composition of one of examples 41-46, further comprising black pepper, vitamin B6, vitamin E, manganese, a cannabinoid, nicotine, or a combination thereof.

48. A method of treating a subject, comprising orally administering the methylthioninium salt-containing composition of one of examples 41-47 to the subject.

49. The method of example 48, wherein the methylthioninium salt-containing composition is dosed on a daily basis to pre-emptively treat general cognitive decline.

50. The method of example 48, wherein the methylthioninium salt-containing composition is dosed on a daily basis to treat general cognitive decline.

51. The method of example 48, wherein the methylthioninium salt-containing composition is dosed on a daily basis to treat traumatic brain injury.

52. A methylthioninium salt-containing composition, comprising methylthioninium chloride, a cannabinoid, vitamin B6, vitamin E, and manganese, wherein the methylthioninium chloride and the cannabinoid are present at a weight ratio from about 1:3 to about 1:10, the methylthioninium chloride and the vitamin B6 are present at a weight ratio from about 10:1 to about 2:1, the methylthioninium chloride and the vitamin E are present at a weight ratio from about 1:3 to about 3:1, and the methylthioninium chloride and the manganese metal content are present at a weight ratio from about 10:1 to about 2:1.

53. The methylthioninium salt-containing composition of example 52, wherein the methylthioninium chloride and the cannabinoid are present at a weight ratio from about 1:4 to 1:7.

54. The methylthioninium salt-containing composition of one of examples 52-53, wherein the methylthioninium chloride and the vitamin B6 are present at a weight ratio from about 7:1 to about 3:1.

55. The methylthioninium salt-containing composition of one of examples 52-54, wherein the caffeine and the vitamin E are present at a weight ratio from about 1:2 to about 1:1.

56. The methylthioninium salt-containing composition of one of examples 52-55, wherein the caffeine and the manganese metal content are present at a weight ratio from about 7:1 to about 2.5:1

57. The methylthioninium salt-containing composition of one of examples 52-56, wherein the composition is in an oral dosage form selected from tablet, capsule, soft-gel, powder, liquid suspension or solution, or transmucosal oral formulation.

58. The methylthioninium salt-containing composition of one of examples 52-57, wherein a single dose of the methylthioninium salt-containing composition includes about 1 mg to about 12 mg of methylthioninium chloride, about 10 mg to about 50 mg of the cannabinoid, about 0.2 mg to about 3 mg of the vitamin B6, about 3 mg to about 15 mg of the vitamin E, and about 0.5 mg to about 4 mg of the manganese metal content.

59. The methylthioninium salt-containing composition of one of examples 52-58, further comprising caffeine, L-theanine, or a combination thereof.

60. The methylthioninium salt-containing composition of one of examples 52-59, further comprising black pepper, vitamin B6, vitamin E, manganese, a cannabinoid, nicotine, or a combination thereof.

61. A method of treating a subject, comprising orally administering the methylthioninium salt-containing composition of one of examples 52-60 to the subject.

62. The method of example 61, wherein the methylthioninium salt-containing composition is dosed on a daily basis to treat seizures.

63. The method of example 61, wherein the methylthioninium salt-containing composition is dosed on a daily basis to treat epilepsy.

64. The method of example 61, wherein the methylthioninium salt-containing composition is dosed on a daily basis for sleep support.

65. A combination therapy, comprising co-administering two or more methylthioninium salt-containing compositions or administering a composition including all compositional components of the two or more methylthioninium salt-containing compositions to a subject, wherein the two or more methylthioninium salt-containing compositions are independently selected from the group consisting of:
MB1 which includes methylthioninium chloride, caffeine, and L-theanine;
MB2 which includes methylthioninium chloride green tea, guarana, and L-theanine;
MB3 which includes methylthioninium chloride, mineral oxides/chlorides, and hydrogen peroxide;
MB4 which includes methylthioninium chloride, mineral oxides/chlorides, hydrogen peroxide, and colloidal silver;
MB5 which includes methylthioninium chloride and vitamin C;
MB6 which includes methylthioninium chloride, glycine, N-acetylcysteine, and vitamin B3; and
MB7 which includes methylthioninium chloride, CBD, vitamin B6, vitamin E, and manganese.

### Definitions

In describing and claiming the present technology, the following terminology will be used.

The singular forms “a,” “an,” and “the” include plural references unless the context clearly dictates otherwise. Thus, for example, reference to “an additive” includes reference to one or more of such components, “a suspension” includes reference to one or more of such suspensions, and “the mixing” refers to one or more of such mixing steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, “about” refers to a degree of deviation based on experimental error typical for the particular property identified. The latitude provided by the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” expressly includes “exactly,” consistent with the discussion below regarding ranges and numerical data.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the terms “treatment” or “treating” of a condition and/or a disease in a mammal means preventing the condition or disease, that is, avoiding any clinical symptoms of the disease; inhibiting the condition or disease, that is, arresting the development or progression of clinical symptoms; and/or relieving the condition or disease, that is, causing the regression of clinical symptoms and/or healing. In further detail, “treating” can include oral administration of any of the methylthioninium salt-containing compounds described herein to heal a subject of a condition or disease, reduce or ameliorate symptoms of a condition or disease, prevent or protect a subject from experiencing or relapsing relative to a condition or disease, causing a condition or disease to regress or retreat, etc.

“Oral” routes of administration include oral ingestion (swallowing) via capsule, tablet, soft-gel, powder, or liquid, e.g., solution or suspension, and/or via transmucosal oral administration, e.g., buccal, sublabial, sublingual, etc. In some instances, some transmucosal oral delivery may occur prior to or after swallowing the bulk of the oral dosage form.

### EXAMPLES

The following examples illustrate embodiments of the disclosure that are presently best known. However, it is to be understood that the following are only exemplary or illustrative of the application of the principles of the present technology. Numerous modifications and alternative compositions, methods, and systems may be devised by those skilled in the art without departing from the spirit and scope of the present disclosure. The appended claims are intended to cover such modifications and arrangements. Thus, while the present disclosure has been described herein with particularity, the following examples provide further detail in connection with what are presently deemed to be practical embodiments of the disclosure.

The preparation of various methylthioninium salt-containing compositions are exemplified herein (see Examples 1-7 below, which are specific examples in line with the more general compositions identified as MB1-MB7 in Table 1 above). Some of those compositions were evaluated in subjects suffering from various conditions, including conditions related to cognition and physical energy, as well as weight loss (see Examples 8-11). Furthermore, the inclusion of mineral oxides/chlorides with colloidal silver (see Example 12) was evaluated for its effectiveness against SARS-CoV-2 (COVID-19).

### Example 1—Preparation of Methylthioninium Salt-Containing Composition 1 (MB1)

A methylthioninium salt-containing composition (MB1) is prepared by admixing pulverized granules/powder of methylthioninium chloride, caffeine, and L-theanine at a 1:20:40 weight ratio. In further detail, other formulation excipients are included for purposes of forming tablets and/or a granule blend for loading into capsules. The granules are then compressed into tablets or loaded into capsules, with each tablet/capsule including about 2.5 mg methylthioninium chloride, 50 mg caffeine, and 100 mg L-theanine. In some examples, when forming compressed tablets, they may be coated with a wax or polymer applied thereto. It is noted that the various components can be combined in liquid form and dried, or can be delivered as a liquid suspension.

### Example 2—Preparation of Methylthioninium Salt-Containing Composition 2 (MB2)

A methylthioninium salt-containing composition (MB2) is prepared by admixing pulverized granules or a powder of methylthioninium chloride, green tea, guarana, and L-theanine at a 1:10:11.4:40 weight ratio. In further detail, other formulation excipients are included for purposes of forming tablets and/or a granule blend for loading into capsules. The granules are then compressed into tablets or loaded into capsules, with each tablet/capsule including about 2.5 mg methylthioninium chloride, 25 mg of green tea (having 50 wt % caffeine), guarana (having 25 wt % caffeine), and 100 mg L-theanine. In some examples, when forming compressed tablets, they may be coated with a wax or polymer applied thereto. It is noted that the various components can be combined in liquid form and dried, or can be delivered as a liquid suspension.

### Example 3—Preparation of Methylthioninium Salt-Containing Composition 3 (MB3)

A methylthioninium salt-containing composition (MB3) is prepared by admixing pulverized granules or a powder of methylthioninium chloride and a mineral oxide/chloride suspension to form a liquid suspension. In this example, hydrogen peroxide is formed and can be present at about 10 wt % or slightly less, with mineral oxychlorides being detectable as an intermediate molecule during production of the hydrogen peroxide. The methylene blue is added to an aqueous carrier at a concentration of about 10 mg dosage per mL (in a 30 mL bottle). The mineral oxide/chloride suspension, which includes magnesium chloride, potassium chlorate, sodium chloride, and magnesium oxide, is added at about 1.5 vol %. This liquid suspension can be orally administered as a liquid, or can be placed in one or more soft gel capsules.

### Example 4—Preparation of Methylthioninium Salt-Containing Composition 4 (MB4)

A methylthioninium salt-containing composition (MB4) is prepared by admixing methylthioninium chloride, a mineral oxide/chloride suspension, and colloidal silver to form a liquid suspension. In this example, hydrogen peroxide is formed and can be present at about 10 wt % or slightly less, with mineral oxychlorides being detectable as an intermediate molecule during production of the hydrogen peroxide. The methylene blue is added to an aqueous carrier at a concentration of about 10 mg dosage per mL (in a 30 mL bottle). The mineral oxide/chloride suspension, which includes magnesium chloride, potassium chlorate, sodium chloride, magnesium oxide is added at about 1.5 vol %. The colloidal silver (Ag404) in this example is added to the liquid suspension to be present in the final composition at about 20 ppm (elemental silver particles having an average particle size of about 15 nm). This liquid suspension can be orally administered as a liquid, or can be placed in one or more soft gel capsule

A methylthioninium salt-containing composition (MB5) is prepared by admixing pulverized granules or a powder of methylthioninium chloride and vitamin C at about a 1:5 weight ratio. In further detail, other formulation excipients are included for purposes of forming tablets and/or a granule blend for loading into capsules. The granules are then compressed into tablets or loaded into capsules, with each tablet/capsule including about 15 mg methylthioninium chloride and about 75 mg of vitamin C. In some examples, when forming compressed tablets, they may be coated with a wax or polymer applied thereto. It is noted that the various components can be combined in liquid form and dried, or can be delivered as a liquid suspension.

### Example 6—Preparation of Methylthioninium Salt-Containing Composition 6 (MB6)

A methylthioninium salt-containing composition (MB6) is prepared by admixing pulverized granules or a powder of methylthioninium chloride, glycine, N-acetylcysteine (NAC), and vitamin B3 (niacinamide) at a 1:10:10:5 weight ratio. In further detail, other formulation excipients are included for purposes of forming tablets and/or a granule blend for loading into capsules. The granules are then compressed into tablets or loaded into capsules, with each tablet/capsule including about 5 mg methylthioninium chloride, 50 mg glycine, 50 mg N-acetylcysteine, and 25 mg niacinamide. In some examples, when forming compressed tablets, they may be coated with a wax or polymer applied thereto. It is noted that the various components can be combined in liquid form and dried, or can be delivered as a liquid suspension.

### Example 7—Preparation of Methylthioninium Salt-Containing Composition 7 (MB7)

A methylthioninium salt-containing composition (MB7) is prepared by admixing pulverized granules or a powder of methylthioninium chloride, a cannabinoid(s) (CBD), vitamin B6 (pyridoxal 5′ phosphate), vitamin E, and manganese (which can be included in the form of a citrate, gluconate, ascorbate, or amino acid chelate, for example) at a 2.5:25:1:7.5:1.5 weight ratio. The manganese content is based on the metal content. The cannabinoid is provided as dry particulate of a 20 wt % water soluble composition. Other formulation excipients are included for forming tablets and/or a granule blend for loading into capsules. The admixture is then compressed into tablets or loaded into capsules, with each tablet/capsule including about 2.5 mg methylthioninium chloride, 25 mg of cannabinoid(s), 1 mg of vitamin B6, 7.5 mg of vitamin E, and 1.5 mg of manganese (by metal content). In some examples, when forming compressed tablets, they may be coated with a wax or polymer applied thereto. It is noted that the various components can be combined in liquid form and dried, or can be delivered as a liquid suspension. It is noted that the various components can be combined in liquid form and dried, or can be delivered as a liquid suspension.

### Example 8—Treatment of Subjects Using MB1

A methylthioninium salt-containing composition (MB1) prepared in accordance with Example 1 was administered to six subjects for at least 1 week primarily for cognitive conditions, e.g., ADD/ADHD, brain fog, lack of energy, etc., with both quantitative (score range from 1-5, with 5 being the best) and qualitative (user comments) results reported, as shown in Table 2:

TABLE 2

| Subject | Score | |
|---|---|---|
| (#) | (1-5) | User Comments |
| 1 | 5 | After 1 week, subject reported positive benefits in |
| | | treating ADHD symptoms, with improved focus. |
| 2 | 5 | After 3 days, subject reported reduction in brain fog |
| | | and daily exhaustion, greater energy during workouts, |
| | | enhanced focus and ability to stay on task at work. |
| 3 | 5 | Subject reported enhanced focus on task and reduced |
| | | brain fog. |
| 4 | 5 | Subject reported enhanced alertness in the morning as |
| | | well as clarity of thinking. |
| 5 | 5 | Subject reported enhanced energy in preparation for |
| | | workouts as well as a generally enhanced feeling |
| | | both physically and mentally. |
| 6 | 5 | Subject reported it enhanced focus and was not |
| | | disruptive in other areas. |

### Example 9—Treatment of Subjects Using MB2

A methylthioninium salt-containing composition (MB2) prepared in accordance with Example 2 was administered to six subjects for primarily for weight loss support (used along with GLP-1 weight loss medication), while retaining mental performance and energy. Quantitative (weight loss) and qualitative (user comments) results reported, as shown in Table 3:

TABLE 3

| Subject | Weight | |
|---|---|---|
| (#) | Loss (lbs.) | User Comments |
| 1 | 170 | Over 2+ years, subject reported significant |
| | | weight loss over a period of multiple years |
| | | without any adverse effects during weight loss |
| | | period. |
| 2 | 30 | Over 3 months, subject reported improved mental |
| | | performance during weight loss period. |
| 3 | 12 | Over 6 weeks, subject reported more energy and |
| | | ability to control stress eating during weight |
| | | loss period. |
| 4 | 30 | Over a period of months, subject reported a |
| | | general sense of being in good shape and |
| | | feeling great during weight loss period. |

### Example 10—Treatment of Subjects Using MB5

A methylthioninium salt-containing composition (MB5) prepared in accordance with Example 5 was administered to six subjects for at least 1 week primarily for cognitive conditions or mental clarity support, e.g., ADD/ADHD, brain fog, lack of energy and/or focus, etc., with both quantitative (score range from 1-5, with 5 being the best) and qualitative (user comments) results reported, as shown in Table 4:

TABLE 4

| Subject | Score | |
|---|---|---|
| (#) | (1-5) | User Comments |
| 1 | 5 | Subject reported enhanced focus, reduced brain |
| | | fog and enhanced mental clarity, reduction in |
| | | afternoon fatigue. |
| 2 | 5 | Subject reported enhanced focus and improved |
| | | sleep. |
| 3 | 5 | Subject reported that a single tablet was enough |
| | | to enhance mental clarity and focus, with 2 |
| | | tablets providing more enhancement on more |
| | | hectic days. |
| 4 | 5 | Subject reported increased morning focus when |
| | | taken the previous evening (without interfering |
| | | with sleep, due to lack of caffeine). |
| 5 | 5 | After just 2 days, subject reported enhanced |
| | | mental focus and improved study in preparation |
| | | for exams. Additionally, anxiety associated |
| | | with exams was also ameliorated. |
| 6 | 5 | After just 1 week, subject reported that greater |
| | | mental focus and clarity, which was noticeably |
| | | improved at work and with hobbies requiring |
| | | mental clarity. Energy was also improved. |
| 7 | 5 | Subject reported a significant enhancement in |
| | | focus and alertness, with reduced brain fog, |
| | | especially later in the day when brain fog can |
| | | be common. |
| 8 | 5 | After just 1 week, subject reported a feeling |
| | | of a brain “reset” with enhanced focus |
| | | and an enhanced ability to read, retain, and |
| | | understand text without the need to re-read |
| 9 | 5 | Subject reported that brain fog was lifted |
| | | throughout the work day. |
| 10 | 4 | User reported that these tablets with methylene |
| | | blue and vitamin C were easy and convenient to |
| | | take with good results, comparable to |
| | | pharmaceutical grade liquid methylene blue and |
| | | suppositories (which lack the vitamin C). |
| 11 | 4 | Subject reported increase in mental energy. |

### Example 11—Treatment of Subjects Using MB6

A methylthioninium salt-containing composition (MB6) prepared in accordance with Example 6 was administered to six subjects for at least 1 week primarily for cognitive conditions, e.g., ADD/ADHD, brain fog, lack of energy, etc., with both quantitative (score range from 1-5, with 5 being the best) and qualitative (user comments) results reported, as shown in Table 5:

TABLE 5

| Subject | Score | |
|---|---|---|
| (#) | (1-5) | Comments |
| 1 | 5 | Subject who has used methylene blue alone has |
| | | reported that its combination with these |
| | | other compounds, e.g., glycine, NAC, and |
| | | niacinamide, enhanced the effect, providing |
| | | the subject with addition daily energy. |
| 2 | 5 | Subject reported a general increase in health |
| | | and wellness. |
| 3 | 5 | Subject reported restorative benefits and |
| | | after 30 days, noticed an improvement in |
| | | short-term memory. |

### Example 12—Mineral Oxides/Chlorides and Colloidal Silver Against COVID-19 (Sars-Cov-2 Virus)

A clinical study was conducted to evaluate the combination of a liquid suspension (Liquid Suspension 1) of colloidal silver (Ag404) with admixed with a suspension of metal oxide and chlorides (containing magnesium chloride, potassium chlorate, sodium chloride, and magnesium oxide) and its effectiveness for killing COVID-19. Multiple aluminum surfaces (12×12 inches each) were used in the evaluation, which were cleaned and dried and then sprayed with a biological sample that contained the COVID-19 virus until each aluminum surface was well covered. Liquid Suspension 1 was then sprayed on the aluminum surfaces and then at various time periods, e.g., 2 minutes, 10 minutes, 6 hours, and 24 hours, each treated sample was used to generate smears taken from the center of each aluminum surface, which were then evaluated by a clinical technician using standard microbe software. An average microbial kill percentage was determined for each sample after each time frame, all of which exhibited about a 97-98% of the COVID-19 virus being killed. Thus, it was surmised that at about 2 minutes, most of the virus was killed, with only nominal increase (if any) when exposed to Liquid Suspension 1 for longer periods of time.

The active ingredients of Liquid Suspension 1 may be formulated into the methylthioninium salt-containing composition of Example 4 (MB4), for example, in treating the COVID-19 virus in a subject. Past protocols have included intubation, which in some instances exacerbated poor outcomes, including death. As COVID-19 is often associated with diminishing oxygen content to the body (due in part to compromised lungs), the MB4 composition of Example 4 can be used to enhance the oxygen efficiency in the body as well as attack the viral load of COVID-19 without the drawbacks associated with intubation of an infected subject.

It is to be understood that the above-referenced arrangements are illustrative of the application for the principles of the present disclosure. Thus, while the present technology has been described herein in connection with the exemplary embodiments, it will be apparent to those of ordinary skill in the art that numerous modifications and alternative arrangements can be made without departing from the principles and concepts of the disclosure as set forth in the claims.

1. A methylthioninium salt-containing composition, comprising methylthioninium chloride, caffeine, and L-theanine, wherein the methylthioninium chloride and the caffeine are present at a weight ratio from about 1:75 to about 1:4 and the methylthioninium chloride and the L-theanine are present at a weight ratio from about 1:200 to about 1:10, and the caffeine and the L-theanine are present at a weight ratio from about 1:5 to about 1:1, wherein the methylthioninium salt-containing composition is in an oral dosage form.

2. The methylthioninium salt-containing composition of claim 1, wherein the methylthioninium chloride and the caffeine are present at a weight ratio from about 1:25 to 1:15; the methylthioninium chloride and the L-theanine are present at a weight ratio from about 1:50 to 1:25; the caffeine and the L-theanine are resent at a weight ratio from about 1:3 to about 1:1.5; or a combination thereof.

3-5. (canceled)

6. The methylthioninium salt-containing composition of claim 1, comprising green tea, guarana, or both providing at least a portion of the caffeine.

7. (canceled)

8. The methylthioninium salt-containing composition of claim 6, wherein the green tea and the guarana are present at a weight ratio of about 1:1.1 to about 1:5, the methylthioninium chloride and the green tea are present at a weight ratio from about 1:5 to about 1:25, and the methylthioninium chloride and the guarana are present at a weight ratio of about 1:10 to about 1:40.

9. The methylthioninium salt-containing composition of claim 1, wherein a single dose of the methylthioninium salt-containing composition includes about 1 mg to about 12 mg of methylthioninium chloride, about 40 mg to about 225 mg of caffeine, and about 50 mg to about 450 mg of L-theanine; or about 1 in to about 12 mg of methylthioninium chloride about 10 mg to about 120 mg of green tea, about 20 mg to about 220 mg of guarana, and about 50 mg to about 450 mg of L-theanine.

10. (canceled)

11. The methylthioninium salt-containing composition of claim 1, further comprising black pepper, vitamin B6, vitamin E, manganese, a cannabinoid, nicotine, or a combination thereof.

12. A method of treating a subject, comprising orally administering the methylthioninium salt-containing composition of claim 1 to the subject.

13. The method of claim 12, wherein the methylthioninium salt-containing composition is dosed on a daily basis to treat attention disorders selected from attention deficit hyperactivity disorder (ADHD) or attention deficit disorder (ADD), support weight loss, enhance metabolism glucagon-like peptide-1 GLP-1, or a combination thereof.

14-15. (canceled)

16. A methylthioninium salt-containing composition, comprising a liquid suspension for oral delivery including water, methylthioninium chloride, mineral oxides and/or chlorides, and hydrogen peroxide, wherein the methylthioninium chloride and the mineral oxides and/or chlorides are present at a weight ratio of about 100:1 to about 1:4.

17. The methylthioninium salt-containing; composition of claim 16, further comprising colloidal silver.

18. The methylthioninium salt-containing composition of claim 17, wherein the colloidal silver is present at from about 5 ppm to about 50 ppm by weight, the colloidal silver has an average particle size from about 3 nm to about 20 nm, or both.

19. (canceled)

20. The methylthioninium salt-containing composition of claim 16, wherein a single dose of the methylthioninium salt-containing composition is from about 0.5 mL to about 2.5 mL and includes from about 1 mg to about 20 mg of the methylthioninium chloride per mL, and from about 0.2 mg to about 10 mg of the mineral oxides and/or chlorides per mL.

21. The methylthioninium salt-containing composition of claim 16, wherein the mineral oxides and/or chlorides include magnesium chloride.

22. The methylthioninium salt-containing composition of claim 16, wherein the weight ratio of the methylthioninium chloride to the mineral oxides and/or chlorides is from about 40:1 to about 2:1.

23. The methylthioninium salt-containing composition of claim 16, further comprising black pepper, vitamin B6, vitamin E, manganese, a cannabinoid, nicotine, caffeine-L-theanine or a combination thereof.

24. (canceled)

25. A method of treating a subject, comprising orally administering the methylthioninium salt-containing composition of claim 16 to the subject.

26. The method of claim 25, wherein the methylthioninium salt-containing composition is dosed on a daily basis to treat long COVID or vaccine-induced spike protein damage, ameliorate brain fog or dementia, treat Lyme disease or chronic fatigue, or a combination thereof.

27-28. (canceled)

29. A methylthioninium salt-containing composition, comprising methylthioninium chloride and vitamin C at a weight ratio from about 1:2.5 to about 1:7.5, wherein the methylthioninium salt-containing composition is in an oral dosage form.

30. The methylthioninium salt-containing composition of claim 29, wherein the weight ratio is from about 4:1 to about 6:1.

31. The methylthioninium salt-containing composition of claim 29, wherein a single dose of the methylthioninium salt-containing composition includes from about 7.5 mg to about 25 mg of the methylthioninium chloride and from about 50 mg to about 150 mg of the vitamin C.

32. (canceled)

33. The methylthioninium salt-containing composition of claim 29, further comprising black pepper, vitamin B6, vitamin E, manganese, a cannabinoid, nicotine, caffeine, L-theanine, or a combination thereof.

34. (canceled)

35. A method of treating a subject, comprising orally administering the methylthioninium salt-containing composition of claim 29 to the subject.

36. The method of claim 35, wherein the methylthioninium salt-containing composition is dosed on a daily basis to treat early stage cognitive decline, dementia, Alzheimer's disease, or a combination thereof; or wherein the methylthioninium salt-containing composition is dosed on a daily basis for weight loss support.

37-39. (canceled)

40. The method of claim 35, wherein the nethvlthioninium salt-containing composition is coadministered at the same time or at different times during the day with a GLP-1 enhancement medication.

41. A methylthioninium salt-containing composition, comprising methylthioninium chloride, glycine, N-acetylcysteine, and a vitamin B3, wherein the methylthioninium chloride and the glycine are present at a weight ratio from about 1:3 to about 1:25, the methylthioninium chloride and the N-acetylcysteine are present at a weight ratio from about 1:3 to about 1:25, and the methylthioninium chloride and the vitamin B3 are present at a weight ratio from about 1:2 to about 1:12, wherein the methylthioninium salt-containing composition is in an oral dosage form.

42. The methylthioninium salt-containing composition of claim 41, wherein the methylthioninium chloride and the glycine are present at a weight ratio from about 1:5 to about 1:15, the methylthioninium chloride and the N-acetylcysteine are present at a weight ratio from about 1:5 to about 1:15, the glycine and the N-acetylcysteine are present at a weight ratio from about 1:2 to about 2:1 or a combination thereof.

43-44. (canceled)

45. The methylthioninium salt-containing composition of claim 41, wherein the vitamin B3 is in the form of niacinamide, nicotinic acid, or a combination thereof.

46. The methylthioninium salt-containing composition of claim 41, further comprising black pepper, vitamin B6, vitamin E, manganese, a cannabinoid, nicotine, caffeine, L-theanine, or a combination thereof.

47. (canceled)

48. A method of treating a subject, comprising orally administering the methylthioninium salt-containing composition of claim 41 to the subject.

49. The method of claim 48, wherein the methylthioninium salt-containing composition is dosed on a daily basis to pre-emptively treat general cognitive decline, treat general cognitive decline, treat traumatic brain injury, or a combination thereof.

50-51. (canceled)

52. A methylthioninium salt-containing composition, comprising methylthioninium chloride, a cannabinoid, vitamin B6, vitamin E, and manganese, wherein the methylthioninium chloride and the cannabinoid are present at a weight ratio from about 1:3 to about 1:10, the methylthioninium chloride and the vitamin B6 are present at a weight ratio from about 10:1 to about 2:1, the methylthioninium chloride and the vitamin E are present at a weight ratio from about 1:3 to about 3:1, and the methylthioninium chloride and the manganese metal content are present at a weight ratio from about 10:1 to about 2:1, wherein the methylthioninium salt-containing composition is in an oral dosage form.

53. The methylthioninium salt-containing composition of claim 52, wherein the methylthioninium chloride and the cannabinoid are present at a weight ratio from about 1:4 to 1:7; the methylthioninium chloride and the vitamin B6 are present at a weight ratio from about 7:1 to about 3:1; the caffeine and the vitamin E are present at a weight ratio from about 1:2 to about 1:1; the caffeine and the manganese metal content are resent at a weight ratio from about 7:1 to about 2.5:1; or a combination thereof.

54-57. (canceled)

58. The methylthioninium salt-containing; composition of claim 52, wherein a single dose of the methylthioninium salt-containing composition includes about 1 mg to about 12 mg of methylthioninium chloride, about 10 mg to about 50 mg of the cannabinoid, about 0.2 mg to about 3 mg of the vitamin 136, about 3 mg to about 15 mg of the vitamin E, and about 0.5 mg to about 4 mg of the manganese metal content.

59. The methylthioninium salt-containing composition of claim 52, further comprising black pepper, vitamin B6 vitamin E, manganese, a cannabinoid, nicotine, caffeine, L-theanine, or a combination thereof.

60. (canceled)

61. A method of treating a subject, comprising orally administering the methylthioninium salt-containing composition of claim 52 to the subject.

62. The method of claim 61, wherein the methylthioninium salt-containing composition is dosed on a daily basis to treat seizures, treat epilepsy, for sleep support, or a combination thereof.

63-64. (canceled)

65. A combination therapy, comprising co-administering two or more methylthioninium salt-containing compositions or administering a composition including all compositional components of the two or more methylthioninium salt-containing compositions to a subject, wherein the two or more methylthioninium salt-containing compositions are independently selected from the group consisting of:
MB1 which includes methylthioninium chloride, caffeine, and L-theanine or M1B2 which includes methylthioninium chloride, green tea, guarana, and L-theanine;
MB3 which includes methylthioninium chloride, mineral oxides/chlorides, and hydrogen peroxide;
MB4 which includes methylthioninium chloride, mineral oxides/chlorides, hydrogen peroxide, and colloidal silver;
M1B5 which includes methylthioninium chloride and vitamin C;
MB6 which includes methylthioninium chloride, glycine, N-acetylcysteine, and vitamin B3; and
M B7 which includes methylthioninium chloride, CBD, vitamin B6, vitamin E, and manganese.
